(19) United States
(12) Patent Application Publication
(10) Pub. No.: US 2001/0049100 A1
(43) Pub. Date: Dec. 6, 2001

(54) Chromosome-based method for facilitating disease diagnosis

(76) Inventor: John F. Stone, Scottsdale, AZ (US)

Correspondence Address: SNELL & WILMER, ONE ARIZONA CENTER, 400 EAST VAN BUREN, PHOENIX, AZ, 850040001

(21) Appl. No.: 09/498,135

(22) Filed: Feb. 4, 2000

(51) Int. Cl. C12Q 1/68; C07H 21/02; C07H 21/04

(52) U.S. Cl. 435/6; 536/23.1

(57) ABSTRACT

A method for facilitating disease diagnosis is disclosed. The method generally includes exposing cells thought to be affected by the disease to a chromosome damaging and/or breakage agent, marking at lease some of the damaged or broken portions of the chromosomes, and analyzing the effect of the chromosome damaging agent.

### TECHNICAL FIELD

The present invention generally relates to disease diagnostic test methods and apparatus. More particularly, the invention relates to inducing and marking chromosome damage to facilitate disease diagnoses.

### BACKGROUND OF THE INVENTION

Certain biochemical changes within cells, which result in damaged chromosomes within the cells or chromosomes which are more sensitive to chromosome damaging agents, are thought to be indicative of or result from particular diseases. For example, it is thought that cells affected by neurodegenerative diseases such as Parkinson's disease and Alzheimer's disease include chromosomes which are either damaged (and not repaired correctly) and/or chromosomes that are hypersensitive to deoxyribonucleic acid (DNA) damaging agents such as ionizing radiation. Thus, chromosome damage or hypersensitivity of chromosomes to DNA damaging agents may be used to detect or diagnose these neurodegenerative diseases.

Diagnosing disease by analyzing chromosome damage or susceptibility to damage may be desirable for several reasons. For example, such diagnosing methods may be useful for testing for diseases which are otherwise generally difficult to diagnose. In particular, such methods may be suitable for diagnosing Alzheimer's disease, which often results in symptoms such as forgetfulness and loss of language, reasoning and understanding skills, and which is diagnosed functionally or symptomatically, through the elimination of other causes of the symptoms associated with the disease. Presently, a diagnosis of Alzheimer's goes unconfirmed until patient death when an autopsy is performed, at which time the diagnosis can be confirmed by the presence of senile plaques and neurofibrillary tangles in the brain tissue. Diagnosing disease via analysis of chromosome damage or susceptibility to damage may also be advantageous because it may allow for early stage or presymptomatic diagnosis of the disease, which in turn may facilitate early administration of any suitable treatment for the disease.

Analyzing chromosome damage or susceptibility to damage to diagnose disease may also be advantageous because such analyses may allow diagnosis of disease which may be caused by genetic factors, sporadic factors, or a combination of factors. For example, Alzheimer's disease, which is thought to result from genetic mutations in about 5-10% of patents with Alzheimer's disease and result from sporadic factors in about 90-95% of patients with Alzheimer's disease, may be diagnosed (both the genetic and sporadic forms of the disease) using chromosome damage analysis techniques. In contrast, typical genetic testing is only suitable for diagnosing Alzheimer's disease in the 5-10% of Alzheimer's patients in which genetic mutations are responsible for the disease symptoms.

A further advantage of analyzing chromosome damage or susceptibility to damage is that such analysis methods may be suitable for diagnosing the onset of the disease (pre or post symptomatically). In comparison, typical genetic testing may only reveal DNA mutations which are indicative of a disease such as Alzheimer's disease; however, the genetic tests are not indicative of whether symptoms of the disease are present (e.g., memory loss in the case of persons with Alzheimer's disease) and do not reveal when such symptoms are likely to arise. In other words, typical genetic tests may be used to determine whether a person has a genetic trait which may be expected to cause disease symptoms at some time during the course of the patient's life, whereas chromosome damage analysis may be used to determine whether a person is presently or about to be affected by the disease.

Recently, methods have been developed to differentiate chromosomes affected by diseases such as Alzheimer's disease and chromosomes that are unaffected by such disease. Such methods generally include application of ultra violet (UV) or gamma radiation to damage the chromosomes, chromosome preparation, and cytogenetic preparation and analysis.

Conventional chromosome comparison techniques are problematic in several regards. In particular, typical chromosome comparison techniques require labor-intensive and relatively expensive UV or gamma radiation application, chromosome preparation, and cytogenetic analysis procedures. The chromosome preparation procedure may be particularly expensive because it requires isolating, manipulating, and analyzing the isolated chromosomes to determine whether the chromosomes were affected by the radiation. Accordingly, improved chromosome comparison techniques suitable for diagnosing disease which are less expensive and less labor intensive are desired.

Another problem with conventional chromosome analysis procedures is that the procedures may be relatively non-specific and insensitive. The conventional analysis methods may be relatively non-specific and insensitive, in part, because the chromosome analysis procedure is relatively expensive and thus only a few cells (e.g., about 100) are typically analyzed at one time. Accordingly, improved methods for analyzing chromosome damage, which facilitate analyzing many cells, are desired.

### SUMMARY OF THE INVENTION

The present invention provides improved techniques suitable for facilitating disease diagnosis. More particularly, the invention provides an improved method to detect spontaneous or induced chromosome breakage, which may be indicative of the presence of the disease.

The way in which the present invention addresses the deficiencies with now-known methods for facilitating disease diagnosis is discussed in greater detail below. However, in general, the present invention provides a relatively easy to perform and relatively inexpensive method for detecting damaged chromosomes or chromosomes hypersensitive to chromosome damaging agents. In addition, the invention provides a cost effective method to test a relatively large number of cells, making the test relatively selective and sensitive with respect to diagnosing the disease.

In accordance with one exemplary embodiment of the present invention, a method of facilitating disease diagnosis includes exposing cells to a chromosome damage inducing agent, breaking the chromosomes, marking at least some of the broken ends of the chromosomes, and counting the marked chromosome pieces. Cells affected by particular diseases are thought to be more susceptible to chromosome breakage upon exposure to the damage inducing agent and subsequent processing. Therefore, diagnosis of a disease may be facilitated by counting a number of cells affected by the agent, a number of marked chromosomes, or a number of marked chromosome pieces.

In accordance with a further exemplary embodiment of the present invention, broken ends of the chromosomes are marked with a luminescent material to facilitate counting of the chromosome pieces. In accordance with one aspect of this embodiment, a number of marked chromosome pieces may be counted using automated photometric analysis equipment.

### BRIEF DESCRIPTION OF THE DRAWINGS

FIG. 1 illustrates a process for damaging and marking chromosomes in accordance with the present invention;

FIG. 2 illustrates a chromosome damage and breakage mechanism; and

FIG. 3 illustrates an alternative chromosome damage and breakage mechanism.

### DETAILED DESCRIPTION OF EXEMPLARY EMBODIMENTS

The present invention generally relates to an assay suitable for facilitating disease diagnosis. More particularly, the invention relates to a chromosome analysis procedure suitable for assisting diagnosis of diseases that render damaged chromosomes or chromosomes that are hypersensitive to chromosome damaging agents.

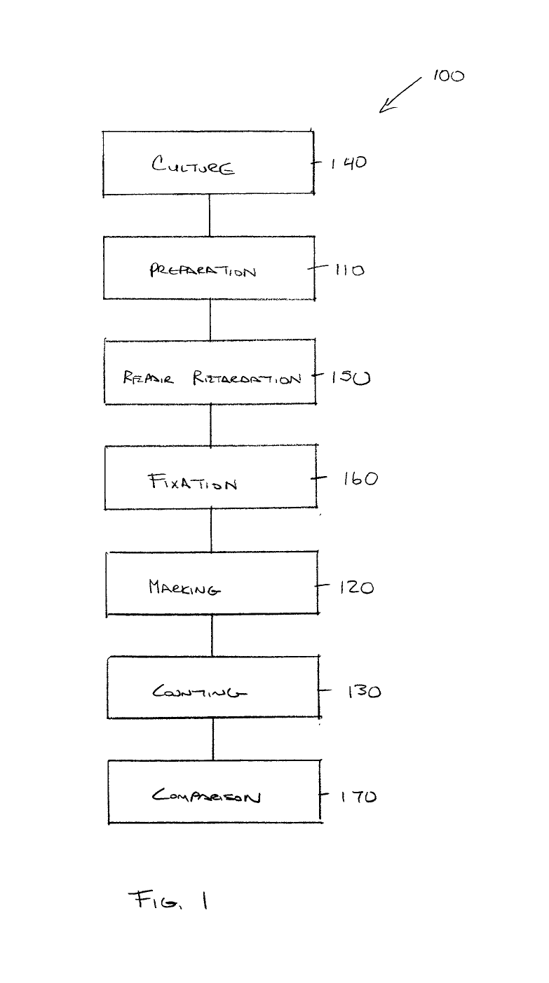

A method 100 for testing cells for a disease in accordance with an exemplary embodiment of the present is illustrated in FIG. 1. Exemplary method 100 includes preparation step 110, a chromosome marking step 120, and a counting step 130. As illustrated in FIG. 1, method 100 may also include a cell culture step 140, an step exposing cells to repair retarding agent 150, a nuclei fixation step 160, and a count comparison step 170.

Culture step 140 generally includes exposing cells to be analyzed to a mitogen to increase a number of cells available for analysis. A preferred type of cell to be cultured in accordance with step 140 may vary in accordance with several factors such as a type of suspected disease, regents used to damage chromosomes within the cells, and the like. However, in accordance with an exemplary embodiment of the present invention, peripheral blood cells are analyzed to test for the presence of the suspect disease (e.g., Alzheimer's disease), and the cells are exposed to phytohemagglutin, pokeweed, or any other suitable mitogen or combination of mitogens configured to cause the cells to divide. In accordance with one exemplary embodiment of the present invention, about 0.5-1.0 ml of blood is mixed with about 5-10 ml of mitogen containing growth medium and the cells are cultured for about 36 to 120 hours.

Preparation step 110 is generally configured to damage or break at least some of the chromosomes of the sample cells in a manner that renders the chromosomes suitable for marking during step 120. More particularly, step 110 is configured to induce greater damage to chromosomes of cells affected by the suspect disease than to cells unaffected by the disease. In accordance with a further aspect of the invention, step 110 is configured to break the damaged chromosomes at or proximate the damaged chromosome sites.

In accordance with an exemplary embodiment of the present invention, the chromosomes are damaged within the nuclei of the sample cells. Thus, expensive, labor intensive chromosome isolation and preparation steps, which are characteristic of conventional chromosome comparison techniques, are not required to facilitate disease diagnosis according to this embodiment of the present invention.

Preparation step 110 may include any technique which facilitates chromosome damage and/or breakage. For example, step 110 may include exposing cells (either cultured cells in accordance with step 140 or uncultured cells) to ionizing radiation (e.g., ultraviolet or gamma radiation) or to one or more chemical agents. In accordance with an exemplary embodiment of the present invention, cells cultured in accordance with step 140 are exposed to a chemical chromosome damaging agent for a period of about 4 to 96 hours. In accordance with exemplary aspects of this embodiment, the damaging agent includes about 0.1 μM to 10 mM 4-nitroquinoline 1-oxide, about 0.1 mM to 1M hydrogen peroxide, or a combination thereof Use of a chemical chromosome damaging agents during step 110 may be particularly advantageous because chemical treatment of the cells, as opposed to radiation treatment, may be well suited for automated assay technology. Automation of step 140 may reduce human labor and labor cost associated with method 100.

In accordance with one embodiment of the present invention, once the chromosomes have been damaged, the chromosomes are broken to facilitate marking of chromosome portions. However, in accordance with alternate embodiments, damaged portions of chromosomes may be marked directly.

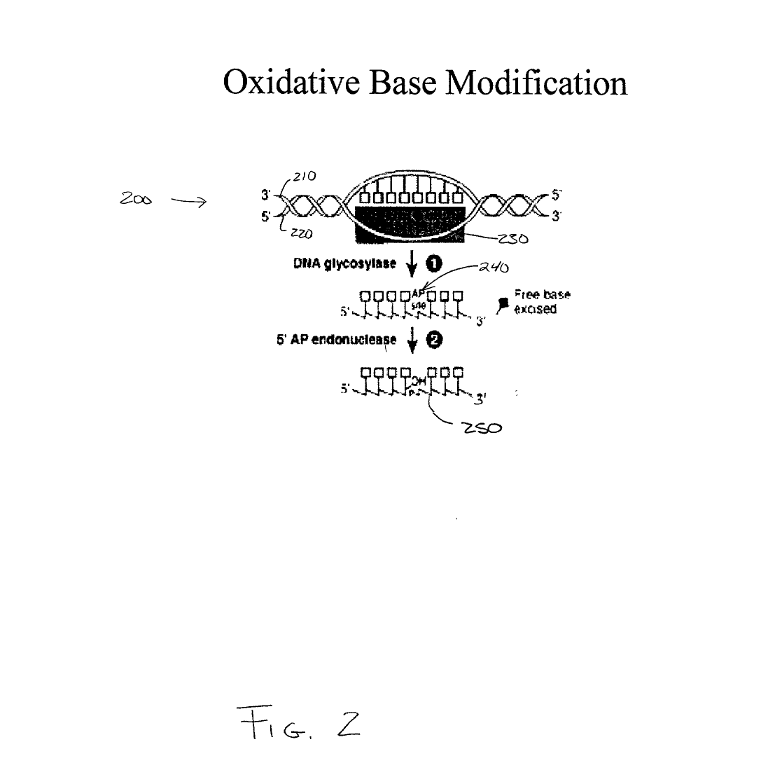

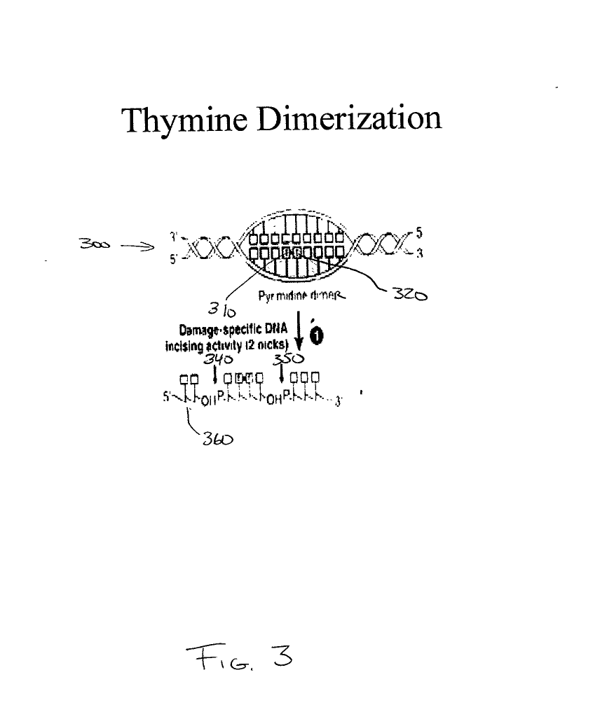

A type of chromosome or breakage mechanism may depend on a variety of factors such as the type of damage inducing agent used in connection with step 110 and the type of disease affecting the cells. Two exemplary breakage mechanisms, namely oxidative base modification and thymine dimerization, are illustrated in FIGS. 2 and 3 respectively. Each of these breakage mechanisms produce 3′—OH DNA strand portions which are well suited for marking during step 120.

As illustrated in FIG. 2, a portion of DNA 200, including a 3′-5′ strand 210 and a 5′-3′ strand 220 may be damaged (e.g., during step 110) by oxidizing a base (e.g. site 230) on either of strands 210 or 220. Oxidized site 230 may then be removed from portion 200 by exposing portion 200 to DNA glycosylase to produce a vacant site 240. An enzyme configured to break a DNA strand at vacant site 240 (e.g., AP endonuclease) may then be employed to produce a 3′—OH strand portion 250 suitable for marking.

A DNA strand (e.g., portion 300) may also be damaged and broken according to the thymine dimerization mechanism illustrated in FIG. 3. Thymine dimerization may occur when a chromosome is exposed to a DNA damaging agent, creating two cross-linked thymine residues (e.g., bases 310 and 320). A DNA portion 330 is then broken proximate the cross-linked thyming groups (e.g., at sites 340 and 350) by an enzymatic mechanism within the cell.

Repair retarding agent exposure step 150 is generally configured to inhibit repair of DNA damage that occurs during step 110. If chromosomes that are damaged during step 110 are left untreated, the chromosomes may be prone to self repair. This DNA self repair may mitigate the effects of step 110, and consequently reduce the sensitivity and selectively of method 100.

Various repair retarding agents may be used to reduce unwanted DNA repair during method 100. In accordance with one exemplary embodiment of the present invention, caffeine is used as the DNA repair inhibiting agent, and DNA repair is inhibited by exposing cultured cells to about 0.2 mM to 20 mM caffeine for about 2-24 hours.

Damaged cell nuclei may suitably be fixed to a medium to facilitate marking and analysis of the damaged chromosomes. For example, in accordance with on exemplary embodiment of the invention, cell nuclei are fixed to a slide using hypotonic swelling and methanol: acetic acid fixation procedures followed by slide preparation. However, other preparation procedures may be employed in accordance with the present invention. For example, other slide preparation, fluid solution preparation or similar techniques, may be used in accordance with the present invention.

After the chromosomes have been prepared in accordance with step 110 and, if desired fixed in accordance with step 160, broken or damaged sites on the chromosomes or portions thereof are marked in accordance with marking step 120. As previously noted, step 120 is generally configured to mark broken or damaged sites or portions of chromosomes to facilitate counting of the damaged or broken sites.

In accordance with an exemplary embodiment of the present invention, 3—OH termini of DNA portions are labeled with fluorescent material such as fluoresceinated dUPT, bisinylated-dUPT, or other labeled deoxynucleotide triphosphate (dNTP) followed by incubation in the presence of fluoresceinated avidin. A kit suitable for labeling 3′—OH DNA termini is commercially available from Intergen, Inc. of Purchase, N.Y. under the product name Apotag™.

After the chromosome pieces have been labeled, e.g., with fluorescent material, a number of marked chromosomes may be counted to determine whether the cells have been affected by the suspect disease. In accordance with a preferred embodiment of the invention, a number of fluorescent sites is counted automatically—e.g., by using cytophotometric image analysis instrumentation. Alternatively, the number of fluorescent sites may be counted manually using a UV light microscope or a number of affected sites may be estimated by measuring or characterizing a brightness of the sample.

A number of marked sites on suspect cells may be compared to a number of marked sites on cells known or thought to be unaffected by the suspect disease during comparison step 170 to increase the accuracy and/or sensitivity of the method of the present invention. In accordance with one embodiment of the invention, suspect and unaffected cells are separately processed through steps 110-160 of procedure 110 and then separately counted and compared during step 170. To improve accuracy of method 100, the suspect and unaffected cells are suitably processed through step 110-170 under similar conditions.

Although the present invention is set forth herein in the context of the appended drawing figures, it should be appreciated that the invention is not limited to the specific form shown. For example, while the invention is conveniently described above in connection with evaluating cells affected by Alzheimer's disease, the present invention may suitably be used to analyze cells thought to be affected by other diseases, particularly neurodegenerative diseases. Various other modifications, variations, and enhancements in the design and arrangement of the method and apparatus set forth herein, may be made without departing from the spirit and scope of the present invention as set forth in the appended claims.

1. A method suitable for facilitating disease diagnosis, the method comprising the steps of:
exposing cells of a suspected diseased patient to a chromosome damaging agent selected to damage chromosomes within the nuclei of the cells to produce chromosome fragments;
marking at least some of the chromosome fragments; and
analyzing the marked chromosome fragments to determine whether the cells were affected by the disease.

2. The method suitable for facilitating disease diagnosis of claim 1, further comprising the step of culturing the cells.

3. The method suitable for facilitating disease diagnosis of claim 2, wherein said culturing step includes exposing the cells to a mitogen for a period of about 36 to about 120 hours.

4. The method suitable for facilitating disease diagnosis of claim 1, further comprising the step of fixing the nuclei of the cells to a slide.

5. The method suitable for facilitating disease diagnosis of claim 1, further comprising the step of exposing the chromosome fragments to a repair retarding agent.

6. The method suitable for facilitating disease diagnosis of claim 1, wherein said exposing step includes producing 3′—OH strands.

7. The method suitable for facilitating disease diagnosis of claim 1, wherein said exposing step includes damaging the chromosomes with a chemical reagent.

8. The method suitable for facilitating disease diagnosis of claim 1, wherein said marking step includes adding fluorescent material to at least some of the chromosome fragments.

9. The method suitable for facilitating disease diagnosis of claim 1, wherein said marking step includes adding dNTP to at least a portion of the chromosome fragments and exposing the fragments to fluoresceinated material.

10. The method suitable for facilitating disease diagnosis of claim 1, wherein said analyzing step includes automatically measuring a number of marked chromosome fragments.

11. A method for analyzing an effect of disease on cells, the method comprising the steps of:
preparing cells suspected of being diseased by exposing the cells to a chromosome breakage agent to form chromosome pieces within nuclei of the cells;
marking at least a portion of the chromosome pieces;
counting a number of marked chromosome pieces.

12. The method for analyzing an effect of disease on cells of claim 11, further comprising the step of exposing the cells to a chromosome repair retarding agent.

13. The method for analyzing an effect of disease on cells of claim 11, further comprising the step of exposing the cells to a chromosome damaging agent.

14. The method for analyzing an effect of disease on cells of claim 11, further comprising the step of comparing cells suspected of being affected by the disease to cells thought not to be affected by the disease.

15. The method for analyzing an effect of disease on cells of claim 11, wherein said exposing step includes forming 3′—OH ends of DNA strands.

16. A method suitable for facilitating diagnosis of Alzheimer's disease, the method comprising the steps of:
exposing cells thought to be affected by Alzheimer's disease to a chromosome damaging agent;
exposing the cells thought to be affected by Alzheimer's disease to a chromosome breakage agent to form chromosome pieces;
marking at lease some of the chromosome pieces; and
measuring an amount of marked chromosome pieces.

17. The method suitable for facilitating diagnosis of Alzheimer's disease of claim 16, the method further comprising the steps of:
exposing cells thought to be unaffected by Alzheimer's disease to a chromosome damaging agent;
exposing the cells thought to be unaffected by Alzheimer's disease to a chromosome breakage agent to form chromosome pieces;
marking at lease some of the chromosome pieces of cells thought to be unaffected by Alzheimer's disease;
measuring an amount of marked chromosome pieces present within the cells thought to be unaffected by disease; and
comparing a number of marked chromosome pieces present in the cells thought to be affected by the disease to a number of marked chromosomes pieces present in the cells thought to be unaffected by the disease.
